(19) United States
(12) Patent Application Publication
(10) Pub. No.: US 2005/0113287 A1
(43) Pub. Date: May 26, 2005

(54) Composition to enhance joint function and repair

(75) Inventor: Michael Nelson, Reno, NV (US)

Correspondence Address: MCHALE & SLAVIN, P.A., 2855 PGA BLVD, PALM BEACH GARDENS, FL, 33410, US

(73) Assignee: Motion Potion, Inc., Palm City, FL (US)

(21) Appl. No.: 10/970,786

(22) Filed: Oct. 20, 2004

Related U.S. Application Data
(60) Provisional application No. 60/513,379, filed on Oct. 21, 2003.

(51) Int. Cl. A61K 31/715; A61K 38/00; A61K 31/7008; A61K 31/737; A61K 31/198

(52) U.S. Cl. 514/2; 514/54; 514/62; 514/562

(57) ABSTRACT

The present invention relates to a composition to enhance joint function, reduce inflammation and homocysteine levels, and repair cartilage. The present invention relates to a nutritional supplement comprising a glucosamine-containing constituent, a chondroitin-containing constituent, methylsulfonylmethane, and at least one sulfur-containing amino acid. A preferred sulfur-containing amino acid is taurine. The nutritional supplement can also include folic acid, vitamins B₆, B₁₂, C. The nutritional supplement can also include chromium and lipoic acid to improve insulin receptor sensitivity.

### REFERENCE TO RELATED APPLICATIONS

This application claims benefit of the filing date of the following provisional patent application: U.S. Patent Application No. 60/513,379, filed Oct. 21, 2003.

### FIELD OF THE INVENTION

This application relates to a composition for repair and reduction of inflammation of joints; particularly to orally consumed compositions capable of reducing inflammation or inflammatory response and promoting enhanced homocysteine detoxification and rejuvenation or replacement of mammalian, predominantly human, connective tissue.

### BACKGROUND OF THE INVENTION

Cartilagenous disorders broadly describe diseases characterized by degeneration or abnormalities of connective tissues manifested by pain, stiffness, diminished function and potential deformity of the affected area. These disorders can be pathological or result from trauma or injury.

Osteoarthritis is one type of cartilagenous disorder resulting from a series of localized degenerative processes typically indicated by pain and reduced joint motion. It is characterized by disruption of articular cartilage and loss of proteoglycans and collagen. The incidence of osteosarthritis increases with age and is common in a majority of humans over 65 year of age.

Rheumatoid arthritis is a distinctly different disease, resulting from a chronic autoimmune disorder believed to originate in the synovium, the tissues surrounding the joints. Similar to osteoarthritis, rheumatoid arthritis also leads to eventual and progressive cartilage destruction.

Glycosaminoglycans (hereinafter GAGs), also known as mucopolysaccharides, have been utilized to treat cartilagenous disorders and other post-athletic activity diseases for years. Glucosamine, which is synthesized from the intake of glucose, is a major component of GAGs. The long chains and lattices of proteoglycans, collagen, and GAGs form articular cartilage and allows for nutrients and other important molecules to transport through the cartilage as blood is not supplied thereto. In healthy mammals, cartilage and other related structures, such as discs, tendons and ligaments are continuously worn away and reformed during normal activity. Insufficient levels of glucosamine in the bloodstream has been associated with delayed repair to cartilage, connective tissue and possibly bone.

Typically, GAG-containing products are orally consumed by combining with some type of a pharmacologically acceptable carrier, such as pills, beverage mixes (ready to drink and powdered drink mixes), bars, and solid dosage forms (tablets, hard capsules, and soft gelatin capsules). By providing GAGs, such as glucosamine hydrochloride, glucosamine sulfate, hyaluronic acid, keratin, heparin, dermatin or N-acetylglucosamine, or various derivatives or combinations thereof known in the art, the repair of cartilage and related structures is known to be facilitated. Additionally, one or more glucosamines have been combined with a chondroitin-constituent, which is also classified as a type of GAG. Chondroitin sulfate is a particularly preferred form of the chondroitin-constituent since it has been known to repair cartilage while providing sulfur.

Sulfur is necessary for the manufacture of proteins used to repair and maintain collagen in joints. Moreover, sulfur is a component of insulin making it important in the regulation of blood sugar in the body.

Methylsulfonylmethane, also known as MSM, is an organic sulfur-containing compound. MSM has been provided as a supplement alone and in combination with GAGs. MSM increases the permeability of cell walls in tissue, thus allowing toxins created by overexertion and/or inflammation to transport out of the cell, while simultaneously allowing essential nutrients to enter into the cell, thereby improving circulation and reducing inflammation in joints.

Thus, a number of methods and compositions have been developed containing a sulfated form of GAGs and/or MSM. However, the sulfur component contained in MSM and sulfated GAGs is usually completely consumed during processing in vivo and have been found to have little impact upon blood sulfate concentrations. Therefore, prior art compositions do not provide sulfur in amounts effective for enhanced joint repair or to regulate insulin levels. Additionally, the regular use of analgesic agents, as is common with older persons, deplete the body of available sulfur thereby reducing the effectiveness of compositions containing sulfated GAGs and/or MSM. Moreover, GAGs being formed of long chains of modified sugars, increase the blood sugar level and may be harmful for people with increased insulin intolerance, such as those with diabetes.

Thus, it is the purpose of the instant invention to provide at least one additional sulfur containing amino acid, oligopeptide or polypeptide in combination with at least one GAG to adequately increase blood concentrations of the sulfate anion. Such increases in blood sulfate concentrations have been found to ameliorate the salutary and therapeutic effects of GAGs used for joint health. It has been discovered that use of at least one sulfur-containing compound can counteract the blood sugar increasing effects of GAGs. Additionally, the supplement of the present invention may include alpha-lipoic acid, also known as thioctic acid, and/or chromium, both of which have been found to have favorable effects on blood sugar levels in mammals. The combination of a GAG and sulfur-containing compound may be formed as a powder or concentrate and can be reconstituted or blended for consumption utilizing any pharmacologically acceptable carrier, as hereinbefore described.

Studies have found that elevated homocysteine levels are a known inflammatory marker. As such, elevated homocysteine levels are detrimental to articular physiology and vascular health. In addition, persons with elevated homocysteine levels have been shown to have increased incidence of endothelial dysfunction, atherosclerosis and osteoporosis. Elevated homocysteine levels have also been shown to be unfavorable in the repair of cartilage. The instant inventor has discovered that administration of the combination of the present invention in conjunction with vitamins B₁₂, B₆, ascorbic acid, (known as vitamin C), and folic acid, (also known as folate), is effective to decrease homocysteine levels in individuals.

### DESCRIPTION OF THE PRIOR ART

Many studies, literature articles and patents have been directed toward the oral, or even intravenous administration of glycosaminoglycans supplements, such as glucosamine and/or chondroitin, particularly regarding their use in degenerative afflictions in the joints of mammals.

For example, WIPO Publication No. 03/101225 to Lähteenmäki, herein incorporated by reference in its entirety, is drawn to a drink composition and method for composing a drink to be used during long-lasting sports activities. This publication teaches an energy drink composition having favorable effects on sugar metabolism, digestion, joints, bones, cardiovascular system, skin, nails, hormones and the immune system. The drink composition contains, inter alia, an effective amount of one or more of the following substances or substance groups: sugars including glucose, fructose; caffeine; MSM; amino acids including tryptophan or taurine; glycosaminoglycans; vitamin C and B-group vitamins. However, Lähteenmäki fails to teach or suggest a composition containing a combination of exogenous amounts of sulfur (provided by a sulfur-containing amino acid or sulfur-containing oligopeptide) and GAGs in amounts effective to increase sulfate concentration in the synovial fluid and to counteract the blood sugar increasing effects of the GAGs.

WIPO Publication No. 01/032188 A1 to Madere, herein incorporated by reference, discloses a composition of orally administered nutritional supplements to provide optimal delivery of vital metabolic precursors necessary for the production and repair of cartilage. The composition includes glucosamine potassium, MSM, chondroitin sulfate, glucosamine sulfate, glucosamine hydrochloride, N-Acetyl D-Glucosamine and chelated manganese proteinate, combined to work synergistically as a biocatalyst in the production of articular cartilage. Madere fails to disclose exogenous sulfur supplementation derived from at least one sulfur-containing amino acid or peptide in addition to the other sulfur containing constituents such as, MSM, glucosamine sulfate and chondroitin sulfate.

WIPO Publication No. 99/62524 A1 to Henderson et al, herein incorporated by reference, teach a composition and method for the treatment, repair and reduction of inflammation in connective tissue in mammals. The composition of the invention comprises S-Adenosylmethionine (SAM), and a component selected from an aminosugar or salts thereof e.g., glucosamine or glycosaminoglycan (GAG), or mixtures or fragments thereof. The composition can optionally contain manganese, methyl donors or methyl donor cofactors such as vitamins B₁₂, vitamins B₆, folic acid, dimethylglycine or trimethylglycine. Henderson et al does not teach or suggest the use of a GAG in combination with a sulfur-containing amino acid or peptide for supplying exogenous amounts of sulfur.

U.S. Patent App. Pub. No. US 2004/0071752 to Hornack et al., herein incorporated by reference, discloses a dietary and/or therapeutic supplement comprising glucosamine sulfate and MSM that can additionally include vitamins B₃, B complex, B₁₂, and amino acids including L-taurine. Hornack et al do not teach or suggest the synergistic and therapeutic utility obtained by using a GAG in combination with effective amounts of at least one sulfur-containing amino acid or peptide.

U.S. Patent App. Pub. No. US 2004/0151826 A1 to Milligan, herein incorporated by reference, teaches a meat-based chew product for carnivorous pets to promote the development of healthy joints, facilitate joint repair and/or prevent joint complications, such as hip dysplasia, common in domesticated animals. The meat chew comprising at least one animal meat product (i.e. lamb, turkey, etc), at least one carrying agent (i.e. copolymer, powdered vegetable starches, etc) and may further contain at least one nutriceutical. While these nutriceuticals may comprise MSM, glucosamine, chondroitin, taurine, vitamin C or combinations thereof. This application does not teach or suggest a composition comprising exogenous amounts of at least one sulfur-containing amino acid or sulfur-containing peptide, specifically combined to work synergistically to increase the amount of sulfur concentrations circulating in the blood stream in order to ameliorate the effects of GAGS and other constituents contained in present invention.

U.S. Patent App. Pub. No. US 2003/0180389 A1 to Philips, teach a composition for maintenance and repair in the connective tissues of mammals. Philips teaches that it is well known to use products containing glucosamine, chondroitin and MSM to aid in the protection, maintenance and repair of connective tissue of mammals. Philip teaches the need for a composition that includes glucosamine, chrondroitin sulfate and a source of sulfur to support body processes, specifically the use of MSM as a preferred sulfur source. Philips does not disclose the use of a sulfur containing amino acid as a source of exogenous amounts of sulfur. Additionally, the composition of Philips requires the use of vitamin C, or sources of vitamin C, for its ability to convert MSM into the active sulfur compound used by the body.

U.S. Patent App. Pub. No. US 2003/0069202 A1 to Kern et al, herein incorporated by reference, teach compositions, kits and methods for treating joint function, bone function, cardiac function or inflammation. Specifically, the application disclosure is directed to a food, beverage, pharmaceutical, or over-the-counter dietary supplement products. The composition teaches a first component which can include aminosugars (i.e. glucosamine, or galactosamine), glycosaminoglycans (i.e. chondroitin), MSM, precursors of MSM and mixtures thereof, a second component comprising a cation source and an edible acid source. Similar to the above mentioned prior art, this application does not teach or suggest a composition comprising exogenous amounts of at least one sulfur-containing amino acid or sulfur-containing peptide, specifically combined to work synergistically to increase the amount of sulfur concentrations circulating in the blood stream in order to ameliorate the effects of GAGS and other constituents as taught in the present invention.

### SUMMARY OF THE INVENTION

The present invention provides a composition to enhance joint function and repair that additively or synergistically improves joint pain and reduces the progression of joint disease superior to that from the ingestion of consumable products that provide only glucosamines, chondroitin sulfates, MSM, or any combinations thereof.

Accordingly, it is a principle objective of the instant invention to provide a product which enhances joint function and repair by providing a composition that causes a substantially simultaneous increase in blood concentrations of both a glycosaminoglycan and sulfate, obtained by combining a glycosaminoglycan with a sulfur-containing amino acid or sulfur-containing oligopeptide, to provide enhanced salutary and therapeutic effects in the joints of mammals.

One objective of the invention is to provide a composition that increases the sulfur ion content in the blood by providing at least one sulfur-containing amino acid or peptide, a non-limiting example of which is taurine, as it serves to enhance the insulin receptor sensitivity thereby stabilizing blood glucose metabolism.

It is a still further objective of the invention to provide a composition that also enhances the insulin receptor sensitivity by providing additional substance groups, including MSM, alpha-lipoic acid and/or chromium.

Yet another objective of the present invention provides a composition that provides vitamin B₁₂, B₆ and folic acid to reduce homocysteine and thereby reduce inflammation of joint tissue that is attributable to elevated homocysteine levels.

It is an additional objective of the instant invention to decrease the inflammatory cascade of events by including in the composition a combination of vitamins B₁₂, B₆, C, and folic acid. The addition of vitamins B₁₂, B₆, C and folic acid are shown to be of great benefit to those at risk of osteoarthritis, vascular disease, atheroschlerosis, osteroporosis, and heart disease.

It is still a further objective of the instant invention to provide a composition that augments the delivery of sulfate and GAG to the synovial fluid of the joints.

Additionally, another objective of the instant invention is to provide a composition that compensates for the sulfate-depleting effect of an analgesic agent, i.e. acetaminophen, commonly used to treat and manage symptoms associated with acute or chronic joint pain. The present invention provides a composition which is safe for use by younger, as well as older mammals, including humans.

It is a further objective of the instant invention to provide a process for treating the inflammation of joints by administering a glycosaminoglycan and sulfur-containing compound, such that the sulfate ion contained within the sulfur containing compound will assist in the delivery of glycosaminoglycan to said joints.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objectives and features thereof.

### DETAILED DESCRIPTION

The present invention relates generally to a composition to enhance joint function, improve cartilage repair, reduce inflammation and enhance homocysteine detoxification. Specifically, the present invention relates to a composition obtained by combining a glycosaminoglycan with at least one sulfur-containing compound, i.e. a sulfur-containing amino acid, oligopeptide, or polypeptide or combinations thereof, in amounts effective to cause a substantially simultaneous increase in blood concentrations of both a glycosaminoglycan and sulfate ion. In addition, the instant composition may include essential B vitamins (e.g., B₁₂, B₆), vitamin C and folic acid or other compounds known in the art for their reduction of homocysteine levels in vivo. Furthermore, the composition may provide alpha lipoic acid and/or chromium or salts thereof to enhance the sensitivity of the insulin receptor.

The present invention utilizes glycosaminoglycans such as glucosamine. Glucosamine is the most bioavailable GAG found in tissues and cartilage. Glucosamine-containing constituents suitable for use in the present invention include, without limitation, glucosamine sulfate, N-acetylglucosamine, N-acetylgalactosamine, glucosamine hydrochloride, glucosamine hydroiodide, or other salt forms, mixtures or combinations thereof. The most preferred form for the use in the present invention being glucosamine sulfate.

Preferably, the amount of glucosamine is about 100 milligrams (mg) to about 3,000 mg, with about 1,000 mg to about 2,000 mg preferred, and about 1,250 mg to about 1,750 mg more preferred. The amount of glucosamine utilized should be determined appropriately for the product (i.e., capsule, food bar, etc.) being created using this inventive composition. Typically, personal characteristics, including but not limited to, age, weight and/or health dictate the physiologically effective amounts necessary.

Chondroitin is another type of GAG and is a metabolic precursor for articular cartilage. The most preferred form of chondroitin-containing constituents for the use in the present invention being chondroitin sulfate, which is broken down by the body into N-Acetyl galactosamine and sulfate disaccharides. The disaccharide contributing to increased blood sugar levels in vivo.

Preferably, the amount of chondroitin is about 100 mg to about 3,000 mg, with about 1,000 mg to about 2,000 mg preferred, and about 1,250 mg to about 1,750 mg more preferred. The amount of chondroitin utilized should be determined appropriately for the product (i.e., capsule, food bar, etc.) being created using this inventive composition. Typically, personal characteristics, including but not limited to, age, weight and/or health dictate the physiologically effective amounts necessary.

Methylsulfonylmethane, or MSM, is a non-toxic, organic sulfur-containing compound found in a variety of fruits, vegetables, and grains. MSM is believed to support joint function by influencing cellular membrane potentials, that is by allowing toxins created by overexertion, injury and/or inflammation to transport out of the cell, while allowing beneficial nutrients to enter into the cell, thereby improving circulation and reducing inflammation. Due to the fact that the sulfur-component contained in MSM and sulfated GAGs is partially, or totally, lost during processing in vivo it has been discovered that exogenous amounts of sulfur, particularly in the form of a sulfur-containing amino acid, (e.g. taurine) need to be supplied in order for maximum benefits to be attained.

An appropriate sulfur-containing compound can include any sulfur-containing amino acid, including but not limited to all racemic forms of taurine, cysteine, cystine, methionine, or N-acetylcysteine (as known as NAC), or a dipeptide, tripeptide, or tetrapeptide containing one or more mixtures of sulfur-containing amino acids, or any combination of a sulfur-containing amino acid with another sulfur-containing peptide. Preferably, the physiologically effective amount of the sulfur-containing amino acid is about 50 mg to about 2,000 mg, with about 100 mg to about 1000 mg preferred. The amount of sulfur-containing amino acid utilized should be determined appropriately for the product (i.e., capsule, food bar, etc.) being created using this inventive composition.

A additional function of the present composition is the enhanced control of blood sugar level in the body. GAGs are long chain modified sugars and therefore raise the level of sugar in the blood. To counterbalance this, the composition of the present invention contains exogenous amounts of a sulfur containing amino-acid, preferably taurine. Taurine is a particularly preferred form of sulfur containing amino acid since it is known in the art as a powerful antioxidant and counteracts the effects of GAGS in diabetics by controlling the level of blood sugar. High blood sugar has also been found to be a contributory factor in the presence other non cartilagenous disorders, such as Alzheimer's disease. Moreover, taurine serves to preserve cell membrane integrity, further preserving joint function.

Preferably, the amount of the taurine is about 50 mg to about 2,000 mg, with about 100 mg to about 900 mg preferred, and about 500 mg to about 800 mg more preferred. The amount of taurine utilized should be determined appropriately for the product (i.e., capsule, food bar, etc.) being created using this inventive composition.

Additionally, balanced sugar or carbohydrate metabolism may be further enhanced through the addition of a soluble trivalent chromium into the instant composition. In a particularly preferred form, although not limited thereto, chromium picolinate is used. Trivalent chromium forms part of a compound in the body known as glucose tolerance factor (GTF), which is involved in regulating insulin receptor sensitivity. Specifically, chromium has favorable effects on sugar and fatty metabolisms in humans as it serves as a means to block excessive glucose in the blood, thus increasing glucose tolerance. Preferably, the physiologically effective amount of chromium is about 25 μg to about 1000 μg. The amount of chromium utilized should be determined appropriately for the product (i.e., capsule, food bar, etc.) being created using this inventive composition.

Additionally, the present invention can include alpha-lipoic acid, also known as thioctic acid, which is a powerful antioxidant and known to improve insulin sensitivity. Lipoic acid is a naturally occurring compound found in plants and animals. Alpha-lipoic acid containing inter alia two sulfur molecules that are readily oxidized or reduced. Specifically, alpha-dihydrolipoic acid is the reduced form of alpha-lipoic acid and has the unique capacity to regenerate other antioxidants, for example alpha-tocopherol (known as vitamin E) already oxidized to their active or reduced form.

The present invention can include a combination of B-group vitamins, i.e. B₁₂, B₆, and folic acid along with Vitamin C to reduce the known inflammatory marker, homocysteine. These additives may optionally be included in the composition of the instant invention to provide methylation, which facilitates conversion of homocysteine to harmless methionine. Vitamin B₁₂ can be present as methyl-cobalamin, cyanocobalamin or combinations thereof known in the art. Preferably, the physiologically effective amount of the vitamin B₁₂ is about 10 μg to about 100 μg, with about 50 μg to about 150 μg preferred, and about 75 μg more preferred. The amount of vitamin B₁₂ utilized should be determined appropriately for the product (i.e., capsule, food bar, etc.) being created using this inventive composition.

Vitamin B₆ can be present in the instant invention as pyridoxine, pyridoxal-5-phospate, or combinations thereof. Vitamin B₆ can also be provided to act in concert with the inventive composition to reduce homocysteine levels. Preferably, the physiologically effective amount of the vitamin B₆ is about 5 mg to about 40 mg, with about 20 mg to 30 mg preferred, and 25 mg more preferred. The amount of vitamin B₆ utilized should be determined appropriately for the product (i.e., capsule, food bar, etc.) being created using this inventive composition.

Vitamin C is water-soluble vitamin which serves to protect fat-soluble vitamins A and E and fatty acids from oxidation. Collagen is one of the primary constituents contained in the connective tissues of the mammalian body. In absence of adequate amounts of vitamin C, collagen production is disrupted. Preferably, the physiologically effective amount of the vitamin C is about 60 mg to about 1000 mg, with about 120 mg more preferred. The amount of vitamin C utilized should be determined appropriately for the product (i.e., capsule, food bar, etc.) being created using this inventive composition.

Additionally, folic acid can be added to the composition to diminish the level of homocysteine in the blood. The folic acid can be present as folic acid, folate or combinations thereof known to one of ordinary skill. Preferably, the physiologically effective amount of the folic acid is about 200 μg to about 800 μg, with about 100 μg to about 700 μg preferred, and about 250 μg to about 500 μg more preferred. The amount of folic acid utilized should be determined appropriately for the product (i.e., capsule, food bar, etc.) being created using this inventive composition.

Without limiting the scope of the present invention, suitable biologically active agents for incorporation in a composition produced in accordance with the teachings of the instant invention may include agents such as, metabolic pharmaceuticals, digestive disease remedies, anti-allergic pharmaceuticals, peripheral disease remedies, and circulatory disease remedies. In their broadest sense, the “biologically active agents” of the present invention will include both human and veterinary medicaments, hormones, marker compounds, and the like.

Although not shown in the present example, the dietary supplement of the instant invention can contain additional constituents such as minerals or trace elements (i.e. calcium, copper, iodine, iron, manganese, and zinc), antioxidants (i.e. vitamins A, D, and E), S-Adenosylmethionine, cetyl myristoleate (CM), riboflavin, phosphorous, beta carotene, blue green algae, bioflavonoids, isoflavonoids, herbs, phytonutrients, dietary fiber, protein, additional carbohydrates, sugar and the like for their known contribution in the art.

Additionally, the composition of the present invention can be utilized in various forms, including, but not limited to, ready-to-drink beverages, sport drinks (i.e. GATORADE®, POWERADE®), powdered beverages, enhanced water, tablets, lozenges, suspensions, emulsions, hard-shell encapsulated composition, soft gelatin encapsulated composition, JELL-O®, nutritional snack bars, food bars, gelatins, powdered supplements, breakfast cereal and the like. The composition of the present invention can be utilized in any edible composition that one would ingest in order to reap the benefits of the present invention.

Moreover, the present invention may be prepared as an injectable suspension incorporating the appropriate suspending agent and liquid carriers known in the art.

Moreover, any of the above mentioned forms of the instant invention may contain other suitable flavor additives, not limited to the following or combinations thereof: apricot, blueberry, black cherry, cranberry, raspberry, strawberry, grape, lemon, lime, orange, mango, peach, passion fruit, pineapple, kiwi, watermelon, wild berry and other natural or artificial favors known in the art.

The preferred age groups, which could benefit from ingesting the present invention are in any age group, particularly those individuals 50 years or older, when joint pain due to arthritis first occurs, although the composition is well tolerated in children. Another target group that could benefit from ingesting the present invention are individuals who are overweight.

The composition of the present invention can also be utilized in edible compositions for animals, including dog biscuit, dog food, cat treats, and cat food. The benefits to animals being similar to those observed in humans.

### EXAMPLE

A non-limiting illustrative example is presented herein; the following is only an example and not solely representative of the inventive concepts discussed herein.

A composition of the present invention can be manufactured as a storable powder which is readily reconstituted into a beverage. The beverage having the following active ingredients (amounts are for an 16 fluid ounce (fl. oz.) container):

| Component | Amount | |
|---|---|---|
| GAG | 1500 | mg |
| Taurine | 1000 | mg |
| Vitamin C | 120 | mg |
| Vitamin B₅ | 30 | mg |
| Vitamin B₆ | 75 | μg |
| Vitamin B₁₂ | 18 | μg |
| Folic Acid | 500 | μg |

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures. One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A nutritional supplement for use in a mammal for enhancing joint function and repair in vivo comprising in combination:
a physiologically effective amount of at least one glycosaminoglycan;
a physiologically effective amount of at least one sulfur-containing compound selected from the group consisting of a sulfur-containing amino acid, sulfur-containing oligopeptide or combinations thereof; and
a pharmacologically acceptable carrier therefore;
wherein administration of said combination of sulfur-containing compound and glycosaminoglycan provides exogenous sulfate ion to the bloodstream in amounts sufficient to augment delivery of said glycosaminoglycan to said joints thereby ameliorating joint function and repair.

2. The nutritional supplement of claim 1, wherein said at least one physiologically effective glycosaminoglycan is selected from the group consisting of glucosamine or salts thereof, chrondroitin or salts thereof, N-acetylglucosamine or salts thereof, and N-acetylgalactosamine or salts thereof, or combinations thereof.

3. The nutritional supplement of claim 1, wherein said at least one sulfur-containing amino acid is selected from the group consisting of taurine, cysteine, cystine, N-acetylcysteine, methionine or combinations thereof.

4. The nutritional supplement of claim 1, wherein said at least one sulfur-containing oligopeptide is selected from the group consisting of a dipeptide, a tripeptide, a tetrapeptide, or combinations thereof.

5. The nutritional supplement of claim 1 further comprising methylsulfonylmethane in amounts sufficient to provide additional circulating amounts of sulfate ion effective to increase tissue detoxification.

6. The nutritional supplement of claim 1, further comprising a physiologically effective amount of an insulin receptor sensitivity enhancement agent selected from the group consisting of alpha lipoic acid, chromium salts or combinations.

7. The nutritional supplement of claim 1, wherein said nutritional supplement is in combination with an edible item selected from the group consisting of ready-to drink beverages, powdered beverages, enhanced waters, tablets, hard-shell encapsulated composition, soft gelatin encapsulated composition, cereal, snack bars, food bars, gelatins, and powdered supplements.

8. The nutritional supplement of claim 1, further comprising: physiologically effective amounts folic acid, vitamin C, vitamin B₆, and vitamin B₁₂;
wherein said amounts are effective for reduction of homocysteine levels in vivo.

9. A nutritional supplement for enhancing joint function and repair, comprising in combination:
a physiologically effective amount of a glucosamine-constituent;
a physiologically effective amount of taurine; and
a pharmacologically acceptable carrier;
wherein said taurine provides exogenous amounts of sulfate ion when processed in vivo to augment delivery of said glucosamine-constituent to said joints to ameliorate joint function and repair.

10. The nutritional supplement of claim 9 further comprising methylsulfonylmethane whereby additional exogenous sulfate ion is provided for enhanced tissue detoxification.

11. The nutritional supplement of claim 9, further comprising, physiologically effective amounts folic acid, vitamin C, vitamin B₆, and vitamin B₁₂ for reduction of homocysteine levels.

12. The nutritional supplement of claim 9, further comprising a physiologically effective amount selected from the group consisting of alpha lipoic acid, chromium salts or combinations thereof whereby insulin receptor sensitivity is enhanced.

13. The nutritional supplement of claim 9, wherein the nutritional supplement is present in an edible carrier selected from the group consisting of ready-to drink beverages, powdered beverages, enhanced water, tablets, hard-shell encapsulated composition, soft gelatin encapsulated composition, snack bars, food bars, gelatins, and powdered supplements.

14. A process for enhancing insulin receptor sensitivity and
enhancing joint function in vivo, said method comprising:
administering a nutritional supplement including at least one physiologically effective amount of at least one glycosaminoglycan in combination with a physiologically effective amount of at least one sulfur-containing compound selected from an amino acid, oligopeptide, or combinations thereof; and
whereby said sulfur-containing compound provides exogenous amounts of sulfate ion when processed in vivo to augment delivery of glycosaminoglycan to said joints to ameliorate joint function and repair and enhance insulin receptor sensitivity.

15. The method of claim 14, wherein said at least one sulfur-containing amino acid is selected from the group consisting of taurine, cysteine, cystine, N-acetylcysteine or methionine.

16. The method of claim 14, wherein said at least one sulfur-containing oligopeptide is selected from the group consisting of a dipeptide, a tripeptide, and a tetrapeptide.

17. The method of claim 14, wherein said at least one physiologically effective glycosaminoglycan is selected from the group consisting of glucosamine or salts thereof, chrondroitin or salts thereof, N-acetylglucosamine or salts thereof, N-acetylgalactosamine or salts thereof, or combinations thereof.

18. The method of claim 14, further comprising methylsulfonylmethane to provide exogenous amounts of sulfate ion for enhanced tissue detoxification.

19. The method of claim 14, wherein said nutritional supplement is present in an edible carrier selected from the group consisting of ready-to drink beverages, powdered beverages, enhanced waters, tablets, hard-shell encapsulated composition, soft gelatin encapsulated composition, cereal, snack bars, food bars, gelatins, and powdered supplements.

20. The method of claim 14, further comprising a physiologically effective amount selected from the group consisting of alpha lipoic acid, chromium salts or combinations thereof in order to enhance insulin receptor sensitivity.

21. The method of claim 14, further comprising: physiologically effective amounts of folic acid, vitamin C, vitamin B₆, and vitamin B₁₂ for reduction of homocysteine levels.
